(19) United States
(12) Patent Application Publication
(10) Pub. No.: US 2006/0249381 A1
(43) Pub. Date: Nov. 9, 2006

(54) Cellulosic-based resistance domain for an analyte sensor

(76) Inventors: James R. Petisce, San Diego, CA (US); Kum Ming Woo, San Diego, CA (US); Victor Ha, Saint Louis, MO (US); Melissa Nicholas, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/413,242

(22) Filed: Apr. 28, 2006

Related U.S. Application Data
(60) Provisional application No. 60/678,373, filed on May 5, 2005.

(51) Int. Cl. C12Q 1/00

(52) U.S. Cl. 204/403.06

(57) ABSTRACT

The present invention relates generally to devices for measuring an analyte in a host. More particularly, the present invention relates to devices for measurement of glucose in a host that incorporate a cellulosic-based resistance domain.

### RELATED APPLICATION

This application claims the benefit of priority under 35 U.S.C. § 119(e) to U.S. Provisional Application No. 60/678,373, filed May 5, 2005, the contents of which are hereby incorporated by reference in their entirety.

### FIELD OF THE INVENTION

The present invention relates generally to systems and methods for measuring an analyte in a host.

### BACKGROUND OF THE INVENTION

A variety of sensors are known that use an electrochemical cell to provide output signals by which the presence or absence of an analyte in a sample can be determined. For example in an electrochemical cell, an analyte (or a species derived from it) that is electro-active generates a detectable signal at an electrode, and this signal can be used to detect or measure the presence and/or amount within a biological sample. In some conventional sensors, an enzyme is provided that reacts with the analyte to be measured, and the byproduct of the reaction is qualified or quantified at the electrode. An enzyme has the advantage that it can be very specific to an analyte and also, when the analyte itself is not sufficiently electro-active, can be used to interact with the analyte to generate another species which is electro-active and to which the sensor can produce a desired output. In one conventional amperometric glucose oxidase-based glucose sensor, immobilized glucose oxidase catalyses the oxidation of glucose to form hydrogen peroxide, which is then quantified by amperometric measurement (for example, change in electrical current) through a polarized electrode.

There exists a molar excess of glucose relative to the amount of oxygen in the body; that is, for every free oxygen molecule in extracellular fluid, there are typically more than 100 glucose molecules present (see Updike et al., Diabetes Care 5:207-21(1982)). However, an immobilized enzyme-based glucose sensor employing oxygen as co-reactant is preferably supplied with oxygen in non-rate-limiting excess in order for the sensor to respond linearly to changes in glucose concentration, while not responding to changes in oxygen concentration. Specifically, when a glucose-monitoring reaction is oxygen limited, linearity is not achieved above minimal concentrations of glucose. Without a semipermeable membrane situated over the enzyme domain to control the flux of glucose and oxygen, a linear response to glucose levels can be obtained only for glucose concentrations of up to about 40 mg/dL. However, in a clinical setting, a linear response to glucose levels is desirable up to at least about 400 mg/dL.

Some prior art membrane systems have been disclosed, for example, hydrophobic and hydrophilic urethanes, that allow for the modulation of relative diffusion rates of oxygen and glucose. However many prior art membrane systems have suffered from complex chemical processing issues, such as, controlled heating, long cure times, issues with solvents in depositions techniques, and the like.

### SUMMARY OF THE INVENTION

A membrane system is needed that modulates oxygen and glucose (or other analyte) diffusion rates and does not have complex chemical mixing or difficult application processes. The membrane systems of preferred embodiments offer such advantages.

Accordingly, in a first aspect, an analyte sensor for measuring a concentration of an analyte in a host is provided, the sensor comprising an electroactive surface and a membrane system disposed thereon, wherein the membrane system comprises a macromolecular resistance domain configured to control a flux of the analyte therethrough, and wherein the macromolecular resistance domain comprises at least one hydrophilic moiety and at least one hydrophobic moiety.

In an embodiment of the first aspect, the hydrophobic moiety is selected from the group consisting of acetyl, butyryl, propionyl, methoxy, ethoxy, and propoxy.

In an embodiment of the first aspect, the macromolecular resistance domain comprises less than about 35 wt. % acetyl.

In an embodiment of the first aspect, the hydrophilic moiety is selected from the group consisting of hydroxyl, carboxymethyl, and carboxyethyl.

In an embodiment of the first aspect, the macromolecular resistance domain comprises at least about 7 wt. % hydroxyl.

In an embodiment of the first aspect, the sensor is configured to measure a concentration of glucose.

In an embodiment of the first aspect, the sensor is a needle sensor configured for transcutaneous insertion into the host.

In an embodiment of the first aspect, the sensor is configured for wholly implanting into the host.

In an embodiment of the first aspect, the sensor is configured for implantation into a host tissue and wherein the resistance domain is configured to interface with the host tissue.

In an embodiment of the first aspect, the resistance domain is a bioprotective barrier configured to protect the sensor from cellular invasion.

In an embodiment of the first aspect, the membrane system is an ionizing radiation-treated membrane system.

In a second aspect, an analyte sensor for measuring an analyte in a host is provided, the sensor comprising an electroactive surface and a membrane system disposed thereon, wherein the membrane system comprises a resistance domain configured to control a flux of the analyte therethrough, and wherein the resistance domain incorporates hydrophilic substituents and hydrophobic substituents to form a macromolecular structure.

In an embodiment of the second aspect, the hydrophobic substituent is selected from the group consisting of acetyl, butyryl, propionyl, methoxy, ethoxy, and propoxy.

In an embodiment of the second aspect, the macromolecular resistance domain comprises less than about 35 wt. % acetyl.

In an embodiment of the second aspect, the hydrophilic substituent is selected from the group consisting of hydroxyl, carboxymethyl, and carboxyethyl

In an embodiment of the second aspect, the macromolecular resistance domain comprises at least about 7 wt. % hydroxyl.

In an embodiment of the second aspect, the sensor is configured to measure glucose.

In an embodiment of the second aspect, the sensor is a needle sensor configured for transcutaneous insertion into the host.

In an embodiment of the second aspect, the sensor is configured for wholly implanting into the host.

In an embodiment of the second aspect, the sensor is configured for implantation into a host tissue and wherein the resistance domain is configured to interface with the host tissue.

In an embodiment of the second aspect, the resistance domain is a bioprotective barrier configured to protect the sensor from cellular invasion.

In an embodiment of the second aspect, the membrane system is an ionizing radiation-treated membrane system.

In a third aspect, a method for forming an analyte sensor for measuring an analyte in a host is provided, the method comprising forming a membrane system on an electroactive surface, wherein the membrane system comprises a resistance domain configured to control a flux of the analyte therethrough, wherein the resistance domain is formed by applying a solution comprising at least one hydrophilic-hydrophobic macromolecule in a solvent system to the electroactive surface.

In an embodiment of the third aspect, the membrane system is formed on the electroactive surface using a deposition technique selected from the group consisting of dip coating, spray coating, spin coating, and casting.

In an embodiment of the third aspect, the resistance domain is formed by dip coating.

In an embodiment of the third aspect, the method further comprises the step of curing the resistance domain for at least about 2 minutes at ambient temperature.

In an embodiment of the third aspect, the resistance domain is formed by dip coating an insertion rate of about 40 inches per minute.

In an embodiment of the third aspect, the resistance domain is formed by dip coating at a dwell time of less than about 1 second.

In an embodiment of the third aspect, the resistance domain is formed by dip coating at a withdrawal rate of about 40 inches per minute.

In an embodiment of the third aspect, the membrane system further comprises an enzyme domain comprising a co-reactant, wherein the co-reactant is capable of undergoing a reaction with the analyte, and wherein the enzyme domain is formed prior to formation of the resistance domain.

In an embodiment of the third aspect, the macromolecule comprises a cellulosic polymer.

In an embodiment of the third aspect, the cellulosic polymer comprises cellulose acetate.

In an embodiment of the third aspect, the cellulosic polymer comprises from about 3 wt. % to about 10 wt. % cellulose acetate.

In an embodiment of the third aspect, the cellulosic polymer comprises about 7 wt. % cellulose acetate.

In a fourth aspect, a method for providing an analyte sensor having a membrane configured to control a flux of an analyte therethrough to an electroactive surface is provided, the method comprising selecting a first hydrophilic-hydrophobic macromolecule having a first hydrophilic content; forming a first membrane system on an electroactive surface, wherein the membrane system comprises a resistance domain configured to control the flux of the analyte therethrough, and wherein the resistance domain is formed from the first hydrophilic-hydrophobic macromolecule; measuring a resistivity of the first membrane system on the electrochemical surface; selecting a second hydrophilic-hydrophobic macromolecule having a second hydrophilic content, wherein the selection is based on the measured resistivity of the first membrane system; and forming a second membrane system on a second electroactive surface, wherein the membrane system comprises a resistance domain configured to control the flux of the analyte therethrough, and wherein the resistance domain is formed from the second hydrophilic-hydrophobic macromolecule, whereby an analyte sensor comprising the second membrane system and the second electroactive surface is obtained.

In an embodiment of the fourth aspect, the second hydrophilic content is higher than the first hydrophilic content and wherein the second membrane system is less resistive to the analyte than the first membrane system.

In an embodiment of the fourth aspect, the second hydrophilic content is lower than the first hydrophilic content and wherein the second membrane system is more resistive to passage therethrough of the analyte than the first membrane system.

In an embodiment of the fourth aspect, a sensitivity of the first membrane system on the electroactive surface is from about 2.5 pA/mg/dL to about 25 pA/mg/dL.

In an embodiment of the fourth aspect, a sensitivity of the second membrane system on the electroactive surface is from about 2.5 pA/mg/dL to about 25 pA/mg/dL.

In a fifth aspect, an electrochemical analyte sensor for measuring a concentration of an analyte in a host is provided, the sensor comprising an electroactive surface and a membrane system disposed thereon, wherein the membrane system comprises an interference domain configured to resist passage therethrough of at least one interfering species, an enzyme domain configured to catalyze a reaction of the analyte with a co-reactant, and a resistance domain configured to control a flux of the analyte therethrough, wherein the resistance domain comprises a cellulosic polymer.

In an embodiment of the fifth aspect, the interference domain is adjacent to the electroactive surface.

In an embodiment of the fifth aspect, the enzyme domain is more distal to the electroactive surface than the interference domain.

In an embodiment of the fifth aspect, the interference domain is more distal to the electroactive surface than the resistance domain.

In an embodiment of the fifth aspect, the interference domain is more distal to the electroactive surface than the enzyme domain.

In an embodiment of the fifth aspect, the resistance domain is more distal to the electroactive surface than the enzyme domain.

In an embodiment of the fifth aspect, the sensor further comprises an electrode domain, wherein the electrode domain is situated between the interference domain and the electroactive surface.

In an embodiment of the fifth aspect, the resistance domain is an ionizing radiation-treated resistance domain, wherein the ionizing radiation is selected from the group consisting of UV, electron beam, gamma, and X-ray radiation.

In an embodiment of the fifth aspect, the cellulosic polymer is selected from the group consisting of cellulose acetate, 2-hydroxyethyl cellulose, cellulose acetate phthalate, cellulose acetate propionate, cellulose acetate butyrate, and cellulose acetate trimellitate.

In an embodiment of the fifth aspect, the cellulosic polymer comprises cellulose acetate.

In an embodiment of the fifth aspect, the cellulose acetate has a molecular weight of less than about 50,000.

In an embodiment of the fifth aspect, the molecular weight of the cellulose acetate is about 38,000.

In an embodiment of the fifth aspect, the cellulose acetate comprises at least about 7 wt. % hydroxyl.

In an embodiment of the fifth aspect, the cellulose acetate comprises about 8.7 wt. % hydroxyl.

In an embodiment of the fifth aspect, the cellulose acetate comprises less than about 35 wt. % acetyl.

In an embodiment of the fifth aspect, the cellulose acetate comprises about 32 wt. % acetyl.

In an embodiment of the fifth aspect, a thickness of the resistance domain is from about 0.05 microns to about 5 microns.

In an embodiment of the fifth aspect, a thickness of the resistance domain is from about 1 to about 3 microns.

In an embodiment of the fifth aspect, the sensor is configured to measure a concentration of glucose.

In an embodiment of the fifth aspect, the sensor is a needle sensor configured for transcutaneous insertion into the host.

In an embodiment of the fifth aspect, the sensor is configured for wholly implanting into the host.

In an embodiment of the fifth aspect, the sensor is configured for implantation into a host tissue and wherein the resistance domain is configured to interface with the host tissue.

In an embodiment of the fifth aspect, the resistance domain is a bioprotective barrier configured to protect the sensor from cellular invasion.

In an embodiment of the fifth aspect, the membrane system is an ionizing radiation-treated membrane system.

In an embodiment of the fifth aspect, the sensor is an ionizing radiation-treated sensor.

In a sixth aspect, an electrochemical analyte sensor for measuring an analyte in a host is provided, the sensor comprising an electroactive surface and a membrane system disposed thereon, wherein the membrane system comprises a resistance domain configured to control a flux of an analyte therethrough, wherein the resistance domain comprises cellulose acetate comprising at least about 7 wt. % hydroxyl.

In an embodiment of the sixth aspect, the cellulose acetate comprises about 8.7 wt. % hydroxyl.

In an embodiment of the sixth aspect, the resistance domain is an ionizing radiation-treated resistance domain, wherein the ionizing radiation is selected from the group consisting of UV, electron beam, gamma, and X-ray radiation.

In an embodiment of the sixth aspect, the cellulose acetate has a molecular weight of less than about 50,000.

In an embodiment of the sixth aspect, a molecular weight of the cellulose acetate is about 38,000.

In an embodiment of the sixth aspect, the cellulose acetate comprises less than about 35 wt. % acetyl.

In an embodiment of the sixth aspect, the cellulose acetate comprises about 32 wt. % acetyl.

In an embodiment of the sixth aspect, a thickness of the resistance domain is from about 0.05 microns to about 5 microns.

In an embodiment of the sixth aspect, a thickness of the resistance domain is from about 1 to about 3 microns.

In an embodiment of the sixth aspect, the membrane system further comprises an enzyme domain configured to catalyze a reaction with the analyte and a co-reactant.

In an embodiment of the sixth aspect, the enzyme domain comprises glucose oxidase.

In an embodiment of the sixth aspect, the membrane system further comprises an interference domain configured to resist passage therethrough of at least one interfering species.

In an embodiment of the sixth aspect, the electroactive surface comprises a working electrode surface and a reference electrode surface, and wherein the interference domain is adjacent to the working reference electrode surface and the reference electrode surface.

In an embodiment of the sixth aspect, the membrane system further comprises an electrode domain.

In an embodiment of the sixth aspect, the sensor is configured to measure a concentration of glucose.

In an embodiment of the sixth aspect, the sensor is a needle sensor configured for transcutaneous insertion into the host.

In an embodiment of the sixth aspect, the sensor is configured for wholly implanting into the host.

In an embodiment of the sixth aspect, the sensor is configured for implantation into a host tissue and wherein the resistance domain is configured to interface with the host tissue.

In an embodiment of the sixth aspect, the resistance domain is a bioprotective barrier configured to protect the sensor from cellular invasion.

In an embodiment of the sixth aspect, the membrane system is a variable frequency microwave-sterilized membrane system.

In an embodiment of the sixth aspect, the membrane system is an ionizing radiation-treated membrane system.

In an embodiment of the sixth aspect, the sensor is an ionizing radiation-sterilized sensor.

In a seventh aspect, an electrochemical analyte sensor for measuring an analyte in a host is provided, the sensor comprising an electroactive surface and a membrane system disposed thereon, wherein the membrane system comprises a resistance domain configured to control a flux of an analyte therethrough, wherein the resistance domain comprises cellulose acetate comprising less than about 35 wt. % acetyl.

In an embodiment of the seventh aspect, the cellulose acetate comprises about 32 wt. % acetyl.

In an embodiment of the seventh aspect, the resistance domain is an ionizing radiation-treated resistance domain, wherein the ionizing radiation is selected from the group consisting of UV, electron beam, gamma, and X-ray radiation.

In an embodiment of the seventh aspect, the cellulose acetate has a molecular weight of less than about 50,000.

In an embodiment of the seventh aspect, a molecular weight of the cellulose acetate is about 38,000.

In an embodiment of the seventh aspect, the cellulose acetate comprises at least about 7 wt. % hydroxyl.

In an embodiment of the seventh aspect, the cellulose acetate comprises about 8.7 wt. % hydroxyl.

In an embodiment of the seventh aspect, a thickness of the resistance domain is from about 0.05 microns to about 5 microns.

In an embodiment of the seventh aspect, a thickness of the resistance domain is from about 1 to about 3 microns.

In an embodiment of the seventh aspect, the membrane system further comprises an enzyme domain configured to catalyze a reaction with the analyte and a co-reactant.

In an embodiment of the seventh aspect, the enzyme domain comprises glucose oxidase.

In an embodiment of the seventh aspect, the membrane system further comprises an interference domain configured to resist passage therethrough of at least one interfering species.

In an embodiment of the seventh aspect, the electroactive surface comprises a working electrode surface and a reference electrode surface, and wherein the interference domain is adjacent to the working reference electrode surface and the reference electrode surface.

In an embodiment of the seventh aspect, the membrane system further comprises an electrode domain.

In an embodiment of the seventh aspect, the sensor is configured to measure a concentration of glucose.

In an embodiment of the seventh aspect, the sensor is a needle sensor configured for transcutaneous insertion into the host.

In an embodiment of the seventh aspect, the sensor is configured for wholly implanting into the host.

In an embodiment of the seventh aspect, the sensor is configured for implantation into a host tissue and wherein the resistance domain is configured to interface with the host tissue.

In an embodiment of the seventh aspect, the resistance domain is a bioprotective barrier configured to protect the sensor from cellular invasion.

In an embodiment of the seventh aspect, the membrane system is an ionizing radiation-treated membrane system.

In an embodiment of the seventh aspect, the sensor is an ionizing radiation-sterilized sensor.

### BRIEF DESCRIPTION OF THE DRAWINGS

FIG. 1A is a perspective view of a continuous analyte sensor, including an implantable body with a membrane system disposed thereon

FIG. 1B is an expanded view of an alternative embodiment of a continuous analyte sensor, illustrating the in vivo portion of the sensor.

FIG. 2A is a schematic view of a membrane system in one embodiment, configured for deposition over the electroactive surfaces of the analyte sensor of FIG. 1A.

FIG. 2B is a schematic view of a membrane system in an alternative embodiment, configured for deposition over the electroactive surfaces of the analyte sensor of FIG. 1B.

FIG. 3A is a graphical representation that illustrates a sensor's response to glucose in one experiment.

FIG. 3B is a graphical representation that illustrates a sensor's response to oxygen in one experiment.

FIG. 4A is a graphical representation that illustrates a sensor's response to glucose in another experiment.

FIG. 4B is a graphical representation that illustrates a sensor's response to oxygen in another experiment.

### DETAILED DESCRIPTION OF THE PREFERRED EMBODIMENT

Definitions

In order to facilitate an understanding of the preferred embodiments, a number of terms are defined below.

The term “analyte” as used herein is a broad term, and is to be given its ordinary and customary meaning to a person of ordinary skill in the art (and is not to be limited to a special or customized meaning), and refers without limitation to a substance or chemical constituent in a biological fluid (for example, blood, interstitial fluid, cerebral spinal fluid, lymph fluid or urine) that can be analyzed. Analytes can include naturally occurring substances, artificial substances, metabolites, and/or reaction products. In some embodiments, the analyte for measurement by the sensing regions, devices, and methods is glucose. However, other analytes are contemplated as well, including but not limited to acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (a metabolite of cocaine); biotimidase; biopterin; c-reactive protein; camitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free B-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosamimidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, 13); lysozyme; mefloquine; netilmicin; phenobarbitone; phenyloin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-I, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain embodiments. The analyte can be naturally present in the biological fluid, for example, a metabolic product, a hormone, an antigen, an antibody, and the like. Alternatively, the analyte can be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), 5-hydroxyindoleacetic acid (FHIAA), and histamine.

The term “host” as used herein is a broad term, and is to be given its ordinary and customary meaning to a person of ordinary skill in the art (and is not to be limited to a special or customized meaning), and refers without limitation to mammals, particularly humans.

The term “continuous (or continual) analyte sensing” as used herein is a broad term, and is to be given its ordinary and customary meaning to a person of ordinary skill in the art (and is not to be limited to a special or customized meaning), and refers without limitation to the period in which monitoring of analyte concentration is continuously, continually, and or intermittently (regularly or irregularly) performed, for example, about every 5 to 10 minutes.

The term “substantial” and “substantially” as used herein are broad terms, and are to be given their ordinary and customary meaning to a person of ordinary skill in the art (and are not to be limited to a special or customized meaning), and refer without limitation to a sufficient amount that provides a desired function. For example, the interference domain of the preferred embodiments is configured to resist a sufficient amount of interfering species such that tracking of glucose levels can be achieved, which may include an amount greater than 50 percent, an amount greater than 60 percent, an amount greater than 70 percent, an amount greater than 80 percent, and an amount greater than 90 percent of interfering species.

The term “domain” as used herein is a broad term, and is to be given its ordinary and customary meaning to a person of ordinary skill in the art (and is not to be limited to a special or customized meaning), and refers without limitation to a region of the membrane system that can be a layer, multiple layers, a uniform or non-uniform gradient (for example, an anisotropic region of a membrane), or a portion of a membrane.

The phrase “distal to” as used herein is a broad term, and is to be given its ordinary and customary meaning to a person of ordinary skill in the art (and is not to be limited to a special or customized meaning), and refers without limitation to the spatial relationship between various elements in comparison to a particular point of reference. In general, the term indicates an element is located relatively far from the reference point than another element.

The term “proximal to” as used herein is a broad term, and is to be given its ordinary and customary meaning to a person of ordinary skill in the art (and is not to be limited to a special or customized meaning), and refers without limitation to the spatial relationship between various elements in comparison to a particular point of reference. In general, the term indicates an element is located relatively near to the reference point than another element.

The term “in vivo portion” as used herein is a broad term, and is to be given its ordinary and customary meaning to a person of ordinary skill in the art (and is not to be limited to a special or customized meaning), and refers without limitation to the portion of the device (for example, a sensor) adapted for insertion into and/or existence within a living body of a host.

The term “ex vivo portion” as used herein is a broad term, and is to be given its ordinary and customary meaning to a person of ordinary skill in the art (and is not to be limited to a special or customized meaning), and refers without limitation to the portion of the device (for example, a sensor) adapted to remain and/or exist outside of a living body of a host.

The terms “cellulosic polymers” and “cellulosic polymers” as used herein are broad terms, and are to be given their ordinary and customary meaning to a person of ordinary skill in the art (and are not to be limited to a special or customized meaning), and refer without limitation to derivatives of cellulose with carboxylic acid anhydride(s) therein. Examples of cellulosic polymers include cellulose acetate, 2-hydroxyethyl cellulose, cellulose acetate phthalate, cellulose acetate propionate, cellulose acetate butyrate, cellulose acetate trimellitate, and the like.

The term “cellulose acetate” as used herein is a broad term, and is to be given its ordinary and customary meaning to a person of ordinary skill in the art (and is not to be limited to a special or customized meaning), and refers without limitation to any of several compounds obtained by treating cellulose with acetic anhydride.

The term “cellulose acetate butyrate” as used herein is a broad term, and is to be given its ordinary and customary meaning to a person of ordinary skill in the art (and is not to be limited to a special or customized meaning), and refers without limitation to any of several compounds obtained by treating cellulose with acetic anhydride and butyric anhydride.

The term “Nafion®” as used herein is a broad term, and is to be given its ordinary and customary meaning to a person of ordinary skill in the art (and is not to be limited to a special or customized meaning), and refers without limitation to DuPont's trademark of a sulfonated tetrafluorethylene polymer modified from Teflon® developed in the late 1960s. In general, Nafion® is a perfluorinated polymer that contains small proportions of sulfonic or carboxylic ionic functional groups.

The term “cross link” and “cross linking” as used herein are broad terms, and are to be given their ordinary and customary meaning to a person of ordinary skill in the art (and are not to be limited to a special or customized meaning), and refer without limitation to joining (adjacent chains of a polymer or protein) by creating covalent bonds. Cross linking can be accomplished by techniques such as thermal reaction, chemical reaction or by providing ionizing radiation (for example, electron beam radiation, UV radiation, or gamma radiation). In preferred embodiments, cross linking utilizes a technique that forms free radicals, for example, electron beam exposure.

The term “ionizing radiation” as used herein is a broad term, and is to be given its ordinary and customary meaning to a person of ordinary skill in the art (and is not to be limited to a special or customized meaning), and refers without limitation to radiation consisting of particles, X-ray beams, electron beams, UV beams, or gamma ray beams, which produce ions in the medium through which it passes.

The term “electrochemically reactive surface” as used herein is a broad term, and is to be given its ordinary and customary meaning to a person of ordinary skill in the art (and is not to be limited to a special or customized meaning), and refers without limitation to the surface of an electrode where an electrochemical reaction takes place. For example, a working electrode measures hydrogen peroxide produced by the enzyme-catalyzed reaction of the analyte detected, which reacts to create an electric current. Glucose analyte can be detected utilizing glucose oxidase, which produces H₂O₂ as a byproduct. H₂O₂ reacts with the surface of the working electrode, producing two protons (2H⁺), two electrons (2e⁻) and one molecule of oxygen (O₂), which produces the electronic current being detected.

The term “membrane system,” as used herein is a broad term, and is to be given its ordinary and customary meaning to a person of ordinary skill in the art (and is not to be limited to a special or customized meaning), and refers without limitation to a permeable or semi-permeable membrane that can be comprised of one or more domains and is typically constructed of materials of a few microns thickness or more, which is permeable to oxygen and is optionally permeable to, e.g., glucose or another analyte. In one example, the membrane system comprises an immobilized glucose oxidase enzyme, which enables a reaction to occur between glucose and oxygen whereby a concentration of glucose can be measured.

The term “sensor” as used herein is a broad term, and is to be given its ordinary and customary meaning to a person of ordinary skill in the art (and is not to be limited to a special or customized meaning), and refers without limitation to the component or region of a device by which an analyte can be quantified.

The terms “interferants” and “interfering species” as used herein are broad terms, and are to be given their ordinary and customary meaning to a person of ordinary skill in the art (and are not to be limited to a special or customized meaning), and refer without limitation to effects and/or species that interfere with the measurement of an analyte of interest in a sensor to produce a signal that does not accurately represent the analyte concentration. In one example of an electrochemical sensor, interfering species are compounds with an oxidation potential that overlap that of the analyte to be measured, thereby producing a false positive signal.

The term “casting” as used herein is a broad term, and is to be given its ordinary and customary meaning to a person of ordinary skill in the art (and is not to be limited to a special or customized meaning), and refers without limitation to a process where a fluid material is applied to a surface or surfaces and allowed to cure or dry. The term is broad enough to encompass a variety of coating techniques, for example, using a draw-down machine (i.e., drawing-down), dip coating, spray coating, spin coating, tampo printing, or the like.

The term “dip coating” as used herein is a broad term, and is to be given its ordinary and customary meaning to a person of ordinary skill in the art (and is not to be limited to a special or customized meaning), and refers without limitation to coating which involves dipping an object or material into a liquid coating substance.

The term “spray coating” as used herein is a broad term, and is to be given its ordinary and customary meaning to a person of ordinary skill in the art (and is not to be limited to a special or customized meaning), and refers without limitation to coating which involves spraying a liquid coating substance onto an object or material.

The term “spin coating” as used herein is a broad term, and is to be given its ordinary and customary meaning to a person of ordinary skill in the art (and is not to be limited to a special or customized meaning), and refers without limitation to a coating process in which a thin film is created by dropping a raw material solution onto a substrate while it is rotating.

The terms “solvent” and “solvent systems” as used herein are broad terms, and are to be given their ordinary and customary meaning to a person of ordinary skill in the art (and are not to be limited to a special or customized meaning), and refer without limitation to substances (e.g., liquids) capable of dissolving or dispersing one or more other substances. Solvents and solvent systems can include compounds and/or solutions that include components in addition to the solvent itself.

The term “hydrophobic” as used herein is a broad term, and is to be given its ordinary and customary meaning to a person of ordinary skill in the art (and is not to be limited to a special or customized meaning), and refers without limitation to a substance that is only weakly attracted to water. In the context of the preferred embodiments, the term hydrophobic is relative to other substituents or moieties in a particular membrane or domain. For example, a substituent or moiety is hydrophobic if it is more hydrophobic than the other (e.g., hydrophilic) substituent or moiety.

The term “hydrophilic” as used herein is a broad term, and is to be given its ordinary and customary meaning to a person of ordinary skill in the art (and is not to be limited to a special or customized meaning), and refers without limitation to a substance that is strongly attracted to water. In the context of the preferred embodiments, the term hydrophilic is relative to other substituents or moieties in a particular membrane or domain. For example, a substituent or moiety is hydrophilic if it is more hydrophilic than the other (e.g., hydrophobic) substituent or moiety.

The term “macromolecule” as used herein is a broad term, and is to be given its ordinary and customary meaning to a person of ordinary skill in the art (and is not to be limited to a special or customized meaning), and refers without limitation to a molecule of high relative molecular mass, the structure of which essentially comprises the multiple repetition of a number of constitutional units.

The term “moiety” as used herein is a broad term, and is to be given its ordinary and customary meaning to a person of ordinary skill in the art (and is not to be limited to a special or customized meaning), and refers without limitation to a part of a molecule.

The term “substituent” as used herein is a broad term, and is to be given its ordinary and customary meaning to a person of ordinary skill in the art (and is not to be limited to a special or customized meaning), and refers without limitation to an atom or group of atoms that replaces another atom or group in a molecule (or macromolecule).

The terms “sensitivity” and “slope” as used herein are broad terms, and are to be given their ordinary and customary meaning to a person of ordinary skill in the art (and are not to be limited to a special or customized meaning), and refer without limitation to an amount of electrical current produced by a predetermined amount (unit) of the measured analyte. For example, in one preferred embodiment, a sensor has a sensitivity (or slope) of about 3.5 to about 7.5 picoAmps of current for every 1 mg/dL of glucose analyte.

Analyte Sensor

The preferred embodiments relate to the use of an analyte sensor that measures a concentration of analyte of interest or a substance indicative of the concentration or presence of the analyte. In some embodiments, the sensor is a continuous device, for example a subcutaneous, transdermal (e.g., transcutaneous), or intravascular device. In some embodiments, the device can analyze a plurality of intermittent blood samples. The analyte sensor can use any method of analyte-sensing, including enzymatic, chemical, physical, electrochemical, spectrophotometric, polarimetric, calorimetric, radiometric, or the like.

The analyte sensor uses any method, including invasive, minimally invasive, and non-invasive sensing techniques, to provide an output signal indicative of the concentration of the analyte of interest. The output signal is typically a raw signal that is used to provide a useful value of the analyte of interest to a user, such as a patient or physician, who can be using the device. Accordingly, appropriate smoothing, calibration, and evaluation methods can be applied to the raw signal and/or system as a whole to provide relevant and acceptable estimated analyte data to the user.

The methods and devices of preferred embodiments can be employed in a continuous glucose sensor that measures a concentration of glucose or a substance indicative of a concentration or a presence of glucose. However, certain methods and devices of preferred embodiments are also suitable for use in connection with non-continuous (e.g., single point measurement or finger stick) monitors, such as the OneTouch® system manufactured by LifeScan, Inc., or monitors as disclosed in U.S. Pat. Nos. 5,418,142; 5,515,170; 5,526,120; 5,922,530; 5,968,836; and 6,335,203. In some embodiments, the glucose sensor is an invasive, minimally-invasive, or non-invasive device, for example a subcutaneous, transdermal, or intravascular device. In some embodiments, the device can analyze a plurality of intermittent biological samples, such as blood, interstitial fluid, or the like. The glucose sensor can use any method of glucose-measurement, including colorimetric, enzymatic, chemical, physical, electrochemical, spectrophotometric, polarimetric, calorimetric, radiometric, or the like. In alternative embodiments, the sensor can be any sensor capable of determining the level of an analyte in the body, for example oxygen, lactase, hormones, cholesterol, medicaments, viruses, or the like.

The glucose sensor uses any method to provide an output signal indicative of the concentration of the glucose. The output signal is typically a raw data stream that is used to provide a value indicative of the measured glucose concentration to a patient or doctor, for example.

One exemplary embodiment described in detail below utilizes an implantable glucose sensor. Another exemplary embodiment described in detail below utilizes a transcutaneous glucose sensor.

In one alternative embodiment, the continuous glucose sensor comprises a transcutaneous sensor such as described in U.S. Pat. No. 6,565,509 to Say et al. In another alternative embodiment, the continuous glucose sensor comprises a subcutaneous sensor such as described with reference to U.S. Pat. No. 6,579,690 to Bonnecaze et al. or U.S. Pat. No. 6,484,046 to Say et al. In another alternative embodiment, the continuous glucose sensor comprises a refillable subcutaneous sensor such as described with reference to U.S. Pat. No. 6,512,939 to Colvin et al. In another alternative embodiment, the continuous glucose sensor comprises an intravascular sensor such as described with reference to U.S. Pat. No. 6,477,395 to Schulman et al. In another alternative embodiment, the continuous glucose sensor comprises an intravascular sensor such as described with reference to U.S. Pat. No. 6,424,847 to Mastrototaro et al. All of the above patents are incorporated in their entirety herein by reference.

Although a few exemplary embodiments of continuous glucose sensors are illustrated and described herein, it should be understood that the disclosed embodiments are applicable to any device capable of single analyte, substantially continual or substantially continuous measurement of a concentration of analyte of interest and providing an output signal that represents the concentration of that analyte.

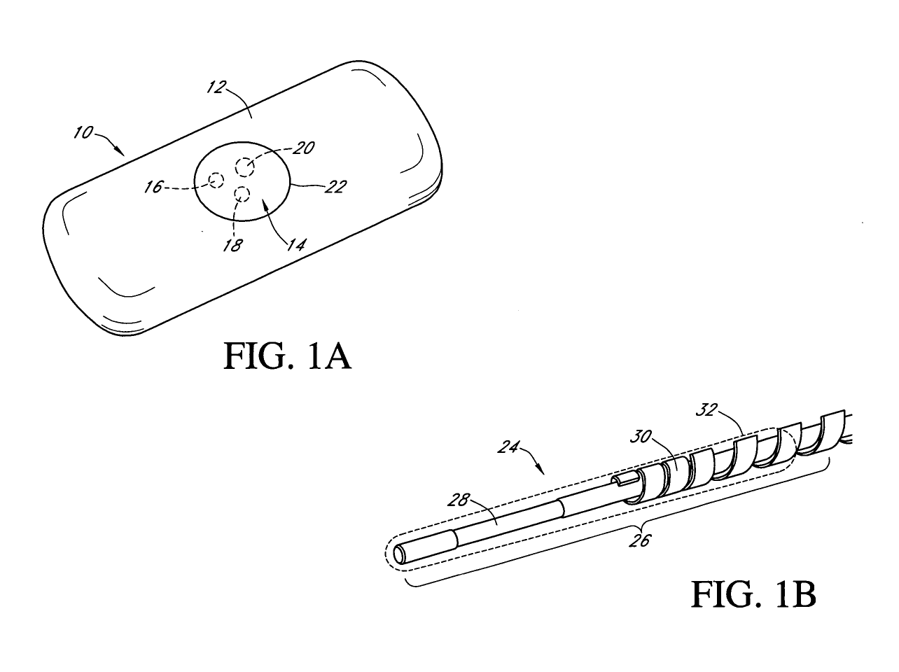

FIG. 1A is a perspective view of an analyte sensor in one embodiment, including an implantable body with a sensing region including a membrane system disposed thereon. In the illustrated embodiment, the analyte sensor 10 includes a body 12 and a sensing region 14 including membrane and electrode systems configured to measure the analyte. Preferably, the sensor 10 is wholly implanted into the subcutaneous tissue of a host, such as described in U.S. Publ. No. US-2006-0015020 A1; U.S. Publ. No. US-2005-0245799-A1; U.S. Publ. No. US-2005-0192557-A1; U.S. Publ. No. US-2004-0199059 A1; U.S. Publ. No. US-2005-0027463-A1; and U.S. Pat. No. 6,001,067.

The body 12 of the sensor 10 can be formed from a variety of materials, including metals, ceramics, plastics, or composites thereof. In one embodiment, the sensor is formed from thermoset molded around the sensor electronics. U.S. Publ. No. US-2004-0199059 A1 discloses suitable configurations for the body.

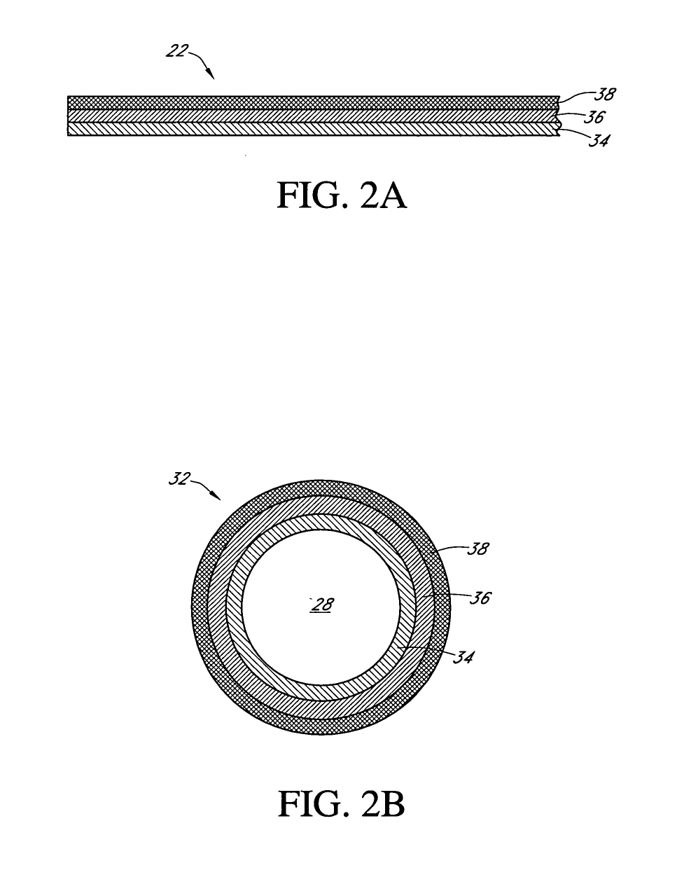

In some embodiments, the sensing region 14 includes a working electrode 16 a reference electrode 18, and a counter electrode 20, which are shown in phantom under membrane system 22. Membrane system 22 is preferably deposited over the electroactive surfaces of the electrodes (16, 18, 20) and includes a plurality of domains or layers, such as described in more detail below, with reference to FIGS. 2A and 2B. In general, the membrane system 22 may be disposed over (deposited on) the electroactive surfaces using methods appreciated by one skilled in the art. See, for example, U.S. Publ. No. US-2006-0015020-A1.

The sensing region 14 comprises electroactive surfaces (the exposed surfaces of electrodes 16, 18, 20), which are in contact with an electrolyte phase (not shown), which is a free-flowing fluid phase disposed between the membrane system 22 and the electroactive surfaces. In this embodiment, the counter electrode 20 is provided to balance the current generated by the species being measured at the working electrode. In the case of glucose oxidase based analyte sensors, the species being measured at the working electrode is H₂O₂. Glucose oxidase catalyzes the conversion of oxygen and glucose to hydrogen peroxide and gluconate according to the following reaction:
Glucose+O₂→Gluconate+H₂O₂

The change in H₂O₂ can be monitored to determine glucose concentration because for each glucose molecule metabolized, there is a proportional change in the product H₂O₂. Oxidation of H₂O₂ by the working electrode is balanced by reduction of ambient oxygen, enzyme generated H₂O₂, or other reducible species at the counter electrode. The H₂O₂ produced from the glucose oxidase reaction further reacts at the surface of the working electrode and produces two protons (2H⁺), two electrons (2e⁻), and one oxygen molecule (O₂). Preferably, one or more potentiostat is employed to monitor the electrochemical reaction at the electroactive surface of the working electrode(s). The potentiostat applies a constant potential to the working electrode and its associated reference electrode to determine the current produced at the working electrode. The current that is produced at the working electrode (and flows through the circuitry to the counter electrode) is substantially proportional to the amount of H₂O₂ that diffuses to the working electrodes. The output signal is typically a raw data stream (measured in “counts”) that can be used to provide a useful value of the measured analyte concentration in a host to the patient or doctor, for example.

FIG. 1B is an expanded view of an alternative exemplary embodiment of a continuous analyte sensor 24, also referred to as a transcutaneous analyte sensor, or needle-type sensor, particularly illustrating the in vivo portion 26 of the sensor. In this embodiment, the in vivo portion of the sensor is the portion adapted for insertion under the host's skin, while an ex vivo portion of the sensor (not shown) is the portion that remains above the host's skin after sensor insertion and operably connects to an electronics unit. In the illustrated embodiment, the analyte sensor 24, includes two electrodes, i.e., a working electrode 28 and at least one additional electrode 30, which may function as a counter and/or reference electrode, hereinafter referred to as the reference electrode 30. Preferably, each electrode is formed from a fine wire, with a diameter in the range of 0.001 to 0.010 inches, for example, and may be formed from plated wire or bulk material, however the electrodes may be deposited on a substrate or other known configurations as is appreciated by one skilled in the art.

In one embodiment, the working electrode 28 comprises a wire formed from a conductive material, such as platinum, palladium, gold, conductive carbon, conductive polymer, or the like. The working electrode 28 is configured and arranged to measure the concentration of an analyte, e.g., glucose. The working electrode 28 is covered with an insulating material, for example a non-conductive polymer. Dip coating, spray coating, vapor-deposition, or other coating or deposition techniques can be used to deposit the insulating material on the working electrode, for example. In one preferred embodiment, the insulating material comprises Parylene, which can be an advantageous conformal coating for its strength, lubricity, and electrical insulation properties, however, a variety of other insulating materials can be used, for example, fluorinated polymers, polyethyleneterephthalate, polyurethane, polyimide, or the like. Preferably, the reference electrode 30, which may function as a reference electrode alone, or as a dual reference and counter electrode, is formed from silver, silver/silver chloride, or the like.

Preferably, the electrodes are juxtapositioned and/or twisted with or around each other; however other configurations are also possible. In one example, the reference electrode 30 is helically wound around the working electrode 28 as illustrated in FIG. 1B. The assembly of wires may then be optionally coated together with an insulating material, similar to that described above, in order to provide an insulating attachment. Some portion of the coated assembly structure is then stripped to expose the necessary electroactive surfaces. In some alternative embodiments, additional electrodes may be included within the assembly, for example, a three-electrode system (including separate reference and counter electrodes) as is appreciated by one skilled in the art.

Preferably, a membrane system 32 is deposited over the electroactive surfaces of the sensor 24 and includes a plurality of domains or layers, such as described in more detail below, with reference to FIGS. 2A and 2B. The membrane system 32 may be deposited on the exposed electroactive surfaces using known thin film techniques (for example, spraying, vapor deposition, electro-depositing, dipping, tampo printing, or the like).

In the illustrated embodiment, the sensor is an enzyme-based electrochemical sensor, wherein the working electrode 16 measures the hydrogen peroxide produced by the enzyme catalyzed reaction of glucose being detected and creates a measurable electronic current (for example, detection of glucose utilizing glucose oxidase produces H₂O₂ peroxide as a by-product, H₂O₂ reacts with the surface of the working electrode producing two protons (2H⁺), two electrons (2e⁻) and one molecule of oxygen (O₂) which produces the electronic current being detected), such as described in more detail above and as is appreciated by one skilled in the art. Preferably, one or more potentiostat is employed to monitor the electrochemical reaction at the electroactive surface of the working electrode(s). The potentiostat applies a constant potential to the working electrode and its associated reference electrode to determine the current produced at the working electrode. The current that is produced at the working electrode (and flows through the circuitry to the counter electrode) is substantially proportional to the amount of H₂O₂ that diffuses to the working electrodes. The output signal is typically a raw data stream that is used to provide a useful value of the measured analyte concentration in a host to the patient or doctor, for example.

U.S. Publ. No. US-2006-0020187-A1 and U.S. Publ. No. US-2006-0015024-A1 disclose system and methods for a transcutaneous analyte sensor that can be incorporated herein with the preferred embodiments.

Some alternative analyte sensors that can benefit from the systems and methods of the preferred embodiments include U.S. Pat. No. 5,711,861 to Ward et al., U.S. Pat. No. 6,642,015 to Vachon et al., U.S. Pat. No. 6,654,625 to Say et al., U.S. Pat. No. 6,565,509 to Say et al., U.S. Pat. No. 6,514,718 to Heller, U.S. Pat. No. 6,465,066 to Essenpreis et al., U.S. Pat. No. 6,214,185 to Offenbacher et al., U.S. Pat. No. 5,310,469 to Cunningham et al., and U.S. Pat. No. 5,683,562 to Shaffer et al., S. Patent 6,579,690 to Bonnecaze et al., U.S. Pat. No. 6,484,046 to Say et al., U.S. Pat. No. 6,512,939 to Colvin et al., U.S. Pat. No. 6,424,847 to Mastrototaro et al., U.S. Pat. No. 6,424,847 to Mastrototaro et al, for example. All of the above patents are incorporated in their entirety herein by reference and are not inclusive of all applicable analyte sensors; in general, it should be understood that the disclosed embodiments are applicable to a variety of analyte sensor configurations.

Membrane System

In general, analyte sensors include a membrane system that functions to control the flux of a biological fluid there through and/or to protect sensitive regions of the sensor from contamination by the biological fluid, for example. Some conventional electrochemical enzyme-based analyte sensors generally include a membrane system that controls the flux of the analyte being measured, protects the electrodes from contamination of the biological fluid, and/or provides an enzyme that catalyzes the reaction of the analyte with a co-factor, for example. See, e.g., U.S. Publ. No. US-2005-0245799-A1 and U.S. Publ. No. US-2006-0020187-A1.

The membrane systems of the preferred embodiments can include any membrane configuration suitable for use with any analyte sensor (such as described in more detail above). In general, the membrane systems of the preferred embodiments includes a plurality of domains, all or some of which can be adhered to or deposited on, over, or around the analyte sensor as is appreciated by one skilled in the art. In one embodiment, the membrane system generally provides one or more of the following functions: 1) protection of the exposed electrode surface from the biological environment, 2) diffusion resistance (limitation) of the analyte, 3) a catalyst for enabling an enzymatic reaction, 4) limitation or blocking of interfering species, and 5) hydrophilicity at the electrochemically reactive surfaces of the sensor interface. Accordingly, the membrane systems of the preferred embodiments include a plurality of domains or layers, for example, an interference domain, an enzyme domain, and a resistance domain, and may include additional domains, such as an electrode domain, a cell disruptive domain, a cell impermeable domain, and/or an oxygen domain (not shown), such as described in more detail elsewhere. However, it is understood that a membrane system modified for other sensors, for example, by including fewer or additional domains is within the scope of the preferred embodiments.

In some embodiments, one or more domains of the membrane systems are formed from materials such as silicone, polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polybutylene terephthalate (PBT), polymethylmethacrylate (PMMA), polyether ether ketone (PEEK), polyurethanes, cellulosic polymers, polysulfones and block copolymers thereof including, for example, di-block, tri-block, alternating, random and graft copolymers. U.S. Publ. No. US-2005-0245799-A1 describes biointerface and membrane system configurations and materials that may be applied to the preferred embodiments.

FIGS. 2A and 2B are schematic views membrane systems in some embodiments that may be disposed over the electroactive surfaces of the analyte sensors of FIGS. 1A and 1B, respectively, wherein the membrane system includes at least a resistance domain, such as described in more detail below. In preferred embodiments, the membrane system of the preferred embodiments additionally includes enzyme and interference domains, such as described in more detail below. However, it is understood that the membrane system of the preferred embodiments can be modified for use in other sensors, by including only one or more of the domains, additional domains not recited above, or for other sensor configurations.

For example, the illustrated embodiment (described below) includes the interference domain disposed most proximal (i.e., adjacent) to the exposed electroactive surfaces, which functions both to block interfering species and to provide hydrophilicity to the exposed electroactive surface thereby enabling electrical communication between the working and reference (and/or counter) electrodes. However, in some alternative embodiments, a separate electrode domain can be deposited on the exposed electroactive surface prior to deposition of the interference domain, for example.

Interference domain

Interferants are molecules or other species that are electro-reduced or electro-oxidized at the electrochemically reactive surfaces, either directly or via an electron transfer agent, to produce a false signal. In preferred embodiments, an interference domain 34 is provided that substantially restricts, resists, or blocks the flow of one or more interfering species. Some known interfering species for a glucose sensor as described in more detail above include acetaminophen, ascorbic acid, bilirubin, cholesterol, creatinine, dopamine, ephedrine, ibuprofen, L-dopa, methyl dopa, salicylate, tetracycline, tolazamide, tolbutamide, triglycerides, and uric acid. In general, the interference domain of the preferred embodiments is less permeable to one or more of the interfering species than to the analyte.

In one embodiment, the interference domain 34 is formed from one or more cellulosic polymers. In general, cellulosic polymers include polymers such as cellulose acetate, cellulose acetate butyrate, 2-hydroxyethyl cellulose, cellulose acetate phthalate, cellulose acetate propionate, cellulose acetate trimellitate, and the like.

In one preferred embodiment, the interference domain 34 is formed from cellulose acetate. Cellulose acetate with a molecular weight of at least about 30,000 to at least about 100,000, preferably from about 35,000, 40,000, or 45,000 to about 55,000, 60,000, 65,000, 70,000, 75,000, 80,000, 85,000, 90,000, or 95,000, and more preferably about 50,000 is preferred. In certain embodiments, however, higher or lower molecular weights can be preferred. Additionally, a casting solution or dispersion of cellulose acetate at a weight percent of at least about 3% to at least about 10%, preferably from about 3.5%, 4.0%, 4.5%, 5.0%, 5.5%, 6.0%, or 6.5% to about 7.5%, 8.0%, 8.5%, 9.0%, or 9.5%, and more preferably about 7% is preferred. A hydroxyl content of cellulose acetate is at least about 2.0% is preferred, and more preferably at least about 3.5%. An acetyl content of cellulose acetate is greater than about 38%, and more preferably greater than about 39.7%. It can be desirable to employ a mixture of cellulose acetates with molecular weights in a single solution, or to deposit multiple layers of cellulose acetate from different solutions comprising cellulose acetates of different molecular weights, different concentrations, or different chemistries. It can also be desirable to include additional substances in the casting solutions or dispersions, e.g., functionalizing agents, crosslinking agents, other polymeric substances, substances capable of modifying the hydrophilicity/hydrophobicity of the resulting layer, and the like.

In some alternative embodiments, additional polymers, such as Nafion®, can be used in combination with cellulosic polymers to provide equivalent and/or enhanced function of the interference domain. In one such exemplary embodiment, a 5 wt % Nafion® casting solution or dispersion is used in combination with a 7 wt % cellulose acetate casting solution or dispersion, for example by dip coating at least one layer of cellulose acetate and subsequently dip coating at least one layer Nafion® onto a needle-type sensor such as described with reference to FIGS. 1B and 2B. Any number of coatings or layers formed in any order may be suitable for forming the interference domain of the preferred embodiments.

In another preferred embodiment, the interference domain 34 is formed from cellulose acetate butyrate. Cellulose acetate butyrate with a molecular weight of about 10,000 daltons to about 75,000 daltons, preferably from about 15,000, 20,000, or 25,000 daltons to about 50,000, 55,000, 60,000, 65,000, or 70,000 daltons, and more preferably about 20,000 daltons is preferred. In certain embodiments, however, higher or lower molecular weights can be preferred. Additionally, a casting solution or dispersion of cellulose acetate butyrate at a weight percent of about 15% to about 25%, preferably from about 15%, 16%, 17%, 18%, 19% to about 20%, 21%, 22%, 23%, 24% or 25%, and more preferably about 18% is preferred. Preferably, the casting solution includes a solvent or solvent system, for example an acetone:ethanol solvent system. Higher or lower concentrations can be preferred in certain embodiments. A plurality of layers of cellulose acetate butyrate can be advantageously combined to form the interference domain in some embodiments, for example, three layers can be employed. It can be desirable to employ a mixture of cellulose acetate butyrate components with different molecular weights in a single solution, or to deposit multiple layers of cellulose acetate butyrate from different solutions comprising cellulose acetate butyrate of different molecular weights, different concentrations, and/or different chemistries (e.g., functional groups). It can also be desirable to include additional substances in the casting solutions or dispersions, e.g., functionalizing agents, crosslinking agents, other polymeric substances, substances capable of modifying the hydrophilicity/hydrophobicity of the resulting layer, and the like.

Additionally, more than one cellulosic polymer can be used to form the interference domain of the preferred embodiments; for example, cellulose acetate and cellulose acetate butyrate can be used together such as described by U.S. Pat. No. 5,520,788. Numerous prior art references describe the use of cellulose acetate and/or Nafion® to block interfering species in a glucose sensor, some of which are cited and incorporated herein by reference in their entirety, including Wilson et al., Analytical Chemistry, 1994, vol. 66, ppl 183-1188; Matsumoto et al., Sensors and Actuators B, 1998, Vol. 49, pp 68-72; Buck et al., Analytica Chimica Acta, 1996, Vol. 319, pp. 335-345; and U.S. Pat. Nos. 3,979,274, 4,073,713, and 5,520,788.

In some alternative embodiments, polyurethanes, polymers having pendant ionic groups, and polymers having controlled pore size, for example, can be utilized as a base material for the interference domain. In one embodiment, the interference domain includes a thin, hydrophobic membrane that is nonswellable in water and restricts diffusion of low molecular weight species. The interference domain 34 is permeable to relatively low molecular weight substances, such as hydrogen peroxide, but restricts the passage of higher molecular weight substances, including glucose and ascorbic acid. Other systems and methods for reducing or eliminating interference species that can be applied to the membrane system of the preferred embodiments are described in U.S. Publ. No. US-2005-0115832-A1; U.S. Publ. No. US-2005-0176136-A1; U.S. Publ. No. US-2005-0161346-A1; and U.S. Publ. No. US-2005-0143635-A1. In some alternative embodiments, a distinct interference domain is not included.

In general, the formation of the interference domain 34 on a surface requires a solvent in order to dissolve the polymer prior to film formation thereon. In one preferred embodiment, acetone and ethanol are used to dissolve cellulose acetate; however one skilled in the art appreciates numerous solvents that are available for cellulosic polymers (and other polymers). Additionally, one skilled in the art appreciates that the relative amounts of solvent may be dependent upon the cellulosic polymer (or other polymer) used, its molecular weight, its method of deposition, and its desired thickness. However, in preferred embodiments, a percent solute from about 1% to about 10% can be used (to form the interference domain solution) to produce the desired result, namely, the interference domain of the preferred embodiments. In fact, it is understood that the cellulosic polymer (or other polymer) used, its molecular weight, method of deposition, and desired thickness are all parameters that may vary depending on one or more other of the parameters, and can be varied accordingly as is appreciated by one skilled in the art.

In preferred embodiments, the interference domain 34 is deposited onto directly onto the electroactive surfaces of the sensor for a domain thickness of from about 0.05 micron or less to about 20 microns or more, more preferably from about 0.05, 0.1, 0.15, 0.2, 0.25, 0.3, 0.35, 0.4, 0.45, 0.5, 1, 1.5, 2, 2.5, 3, or 3.5 to about 4, 5, 6, 7, 8, 9, 10, 11, 12, 13, 14, 15, 16, 17, 18, 19, or 19.5 microns, and more preferably from about 1, 1.5 or 2 microns to about 2.5 or 3 microns. Thicker membranes can also be useful, but thinner membranes are generally preferred because they have a lower impact on the rate of diffusion of hydrogen peroxide from the enzyme membrane to the electrodes. In some alternative embodiments, a distinct electrode domain is formed directly on the exposed electroactive surfaces prior to deposition of the interference domain as described in more detail with reference to U.S. Publ. No. US-2006-0020187-A1.

In general, the membrane system of the preferred embodiments can be formed and/or deposited on the exposed electroactive surfaces (e.g., at least the working and reference electrodes) using known thin film techniques (for example, casting, spray coating, drawing down, vapor deposition, electro-depositing, dip coating, spin coating, tampo printing, and the like). Preferably, the interference domain 34 is deposited by spray or dip coating. In some embodiments, the interference domain 34 is formed by dip coating one or more times (namely, forming one or more of the same or different layers) in a coating solution(s). In embodiments wherein dip coating is used to deposit the interference domain at room temperature, a preferred insertion rate of from about 0.25 inch per minute to about 100 inches per minute, with a preferred dwell time of from about 0 seconds to about 2 minutes, and a preferred withdrawal rate of from about 0.25 inch per minute to about 100 inches per minute provide a functional coating. Suitable cure time ranges from about 1 minute to about 30 minutes at room temperature (and can be accomplished under vacuum (e.g., 20 to 30 in Hg)). However, values outside of those set forth above can be acceptable or even desirable in certain embodiments, for example, dependent upon viscosity and surface tension as is appreciated by one skilled in the art. In one exemplary embodiment of needle-type (transcutaneous) sensor such as described with reference to FIGS. 1B and 2B, a cellulose acetate interference domain 34 is formed by dip coating the sensor into an interference domain solution using an insertion rate of between about 20 and about 60 inches/min., preferably 40 inches/min., a dwell time of between about 0 and about 5 seconds, preferably 0 seconds, and a withdrawal rate of between about 20 and about 60 inches/min., preferably 40 inches/min., and curing the domain for from about 3 minutes at room temperature. The dip process may be repeated at least one time and up to 10 times or more, preferably about 4 times, to form an appropriate domain thickness (e.g., 1-2 microns). However, the interference domain can be formed using any known method as will be appreciated by one skilled in the art.

Enzyme Domain

In preferred embodiments, the membrane system further includes an enzyme domain 36 disposed more distally from the electroactive surfaces than the interference domain 34, however other configurations are possible (e.g., interference domain may be deposited over enzyme domain). In the preferred embodiments, the enzyme domain 36 provides an enzyme to catalyze the reaction of the analyte and its co-reactant, as described in more detail below. In the preferred embodiments of a glucose sensor, the enzyme domain includes glucose oxidase; however other oxidases, for example, galactose oxidase or uricase oxidase, can also be used.

For an enzyme-based electrochemical glucose sensor to perform well, the sensor's response is preferably limited by neither enzyme activity nor co-reactant concentration. Because enzymes, including glucose oxidase, are subject to deactivation as a function of time even in ambient conditions, this behavior is compensated for in forming the enzyme domain. Preferably, the enzyme domain 36 is constructed of aqueous dispersions of colloidal polyurethane polymers including the enzyme. However, in alternative embodiments the enzyme domain is constructed from an oxygen enhancing material, for example, silicone, or fluorocarbon, in order to provide a supply of excess oxygen during transient ischemia. Preferably, the enzyme is immobilized within the domain. See U.S. Publ. No. US-2005-0054909-A1.

In preferred embodiments, the enzyme domain 36 is deposited onto the interference domain for a domain thickness of from about 0.05 micron or less to about 20 microns or more, more preferably from about 0.05, 0.1, 0.15, 0.2, 0.25, 0.3, 0.35, 0.4, 0.45, 0.5, 1, 1.5, 2, 2.5, 3, or 3.5 to about 4, 5, 6, 7, 8, 9, 10, 11, 12, 13, 14, 15, 16, 17, 18, 19, or 19.5 microns, and more preferably from about 2, 2.5 or 3 microns to about 3.5, 4, 4.5, or 5 microns. However in some embodiments, the enzyme domain is deposited onto an optional electrode domain or directly onto the electroactive surfaces. Preferably, the enzyme domain 36 is deposited by spray or dip coating. In one embodiment of needle-type (transcutaneous) sensor such as described with reference to FIGS. 1B and 2B, the enzyme domain 36 is formed by dip coating the interference domain coated sensor into an enzyme domain solution and curing the domain for from about 15 to about 30 minutes at a temperature of from about 40 to about 55° C. (and can be accomplished under vacuum (e.g., 20 to 30 in Hg)). In embodiments wherein dip coating is used to deposit the enzyme domain at room temperature, a preferred insertion rate of from about 0.25 inch per minute to about 3 inches per minute, with a preferred dwell time of from about 0.5 minutes to about 2 minutes, and a preferred withdrawal rate of from about 0.25 inch per minute to about 2 inches per minute provide a functional coating. However, values outside of those set forth above can be acceptable or even desirable in certain embodiments, for example, dependent upon viscosity and surface tension as is appreciated by one skilled in the art. In one embodiment, the enzyme domain 36 is formed by dip coating two times (namely, forming two layers) in an enzyme domain solution and curing at 50° C. under vacuum for 20 minutes. However, in some embodiments, the enzyme domain can be formed by dip coating and/or spray coating one or more layers at a predetermined concentration of the coating solution, insertion rate, dwell time, withdrawal rate, and/or desired thickness.

Resistance Domain

In preferred embodiments, the membrane system includes a resistance domain 38 disposed more distal from the electroactive surfaces than the enzyme domain 36. Although the following description is directed to a resistance domain for a glucose sensor, the resistance domain can be modified for other analytes and co-reactants as well.

The resistance domain 38 includes a semi permeable membrane that controls the flux of oxygen and glucose to the underlying enzyme domain 36, preferably rendering oxygen in a non-rate-limiting excess. As a result, the upper limit of linearity of glucose measurement is extended to a much higher value than that which is achieved without the resistance domain. In one embodiment, the resistance domain 38 exhibits an oxygen to glucose permeability ratio of from about 50:1 or less to about 400:1 or more, preferably about 200:1. As a result, one-dimensional reactant diffusion is adequate to provide excess oxygen at all reasonable glucose and oxygen concentrations found in the subcutaneous matrix (See Rhodes et al., Anal. Chem., 66:1520-1529 (1994)).

In some embodiments, the resistance domain is formed as a macromolecule including at least one hydrophilic moiety and at least one hydrophobic moiety. Some hydrophobic moieties suitable for the resistance domain include acetyl, butyryl, propionyl, methoxy, ethoxy, and propoxy. In one preferred embodiment, the hydrophobic moiety is acetyl; while the preferred macromolecule has less than about 35% acetyl, other concentrations are possible. Some hydrophilic moieties suitable for the resistance domain include hydroxyl, carboxymethyl, and carboxyethyl. In one preferred embodiment, the preferred hydrophilic moiety is hydroxyl; while the preferred macromolecule has more than about 7% hydroxyl, other concentrations are also possible.

In some embodiments, the macromolecule comprises a cellulosic polymer; in one preferred embodiment, the macromolecule comprises cellulose acetate (CA). In one preferred CA resistance domain, the CA resistance domain is formed from CA that has a weight percent of at least about 3% to about 10%, and more preferably about 7%. Suitable thin film deposition techniques and solvents are appreciated by one skilled in the art. In some embodiments, dip coating, spray coating, spin coating, vapor deposition or casting are used to form the membrane domain on a surface (e.g., of the sensor).

A resistance domain formed from a macromolecule as described herein is advantageous because the functionality (e.g., relative resistivity or analyte flux) of the resistance domain can be easily tuned (increased or decreased) by changing the relative amounts of the hydrophobic and hydrophilic moieties or substituents. Namely, macromolecular engineering applied to the resistance domain enables selection and placement of the hydrophilic and hydrophobic moieties onto the same chain (backbone), and thereby forming a resistance domain without special processing or mixing required. In some embodiments, the resistance domain 38 is formed from one or more cellulosic polymers. In general, cellulosic polymers include polymers such as cellulose acetate, cellulose acetate butyrate, 2-hydroxyethyl cellulose, cellulose acetate phthalate, cellulose acetate propionate, cellulose acetate trimellitate, and the like.

In one preferred embodiment, the resistance domain 38 is formed from cellulose acetate. Cellulose acetate with a molecular weight of at least about 30,000 to at least about 100,000, preferably from about 35,000, 40,000, or 45,000 to about 55,000, 60,000, 65,000, 70,000, 75,000, 80,000, 85,000, 90,000, or 95,000, and more preferably about 38,000 is preferred. Additionally, a casting solution or dispersion of cellulose acetate at a weight percent of at least about 3% to at least about 10%, preferably from about 3.5%, 4.0%, 4.5%, 5.0%, 5.5%, 6.0%, or 6.5% to about 7.5%, 8.0%, 8.5%, 9.0%, or 9.5%, and more preferably about 7% is preferred. A hydroxyl content of cellulose acetate is at least about 7% is preferred, and more preferably at least about 8.7%. An acetyl content of cellulose acetate is less than about 35%, and more preferably less than about 32%. In certain embodiments, however, higher or lower molecular weights, acetyl content, and/or hydroxyl content can be preferred. It can be desirable to employ a mixture of cellulose acetates with molecular weights in a single solution, or to deposit multiple layers of cellulose acetate from different solutions comprising cellulose acetates of different molecular weights, different concentrations, or different chemistries. It can also be desirable to include additional substances in the casting solutions or dispersions, e.g., functionalizing agents, crosslinking agents, other polymeric substances, substances capable of modifying the hydrophilicity/hydrophobicity of the resulting layer, and the like. In some embodiments, more than one cellulosic polymer can be used to form the resistance domain of the preferred embodiments; for example, cellulose acetate and cellulose acetate butyrate can be used together.

In general, the cellulosic polymer chosen for the resistance domain of the preferred embodiments can have a hydroxyl content with sufficient hydrophilicity such that glucose permeates through the domain (or membrane system). Additionally, modulating the hydroxyl to acetyl ratio within the cellulosic polymer can be used to modulate the relative permeability of glucose through the resistance domain. For example, it is believed that by increasing the hydroxyl concentration an increased slope (i.e., glucose permeability) can be achieved. The ability to easily and reliably modulate the relative diffusivity of the resistance domain to the analyte (and its co-reactant) is advantageous as is appreciated by one skilled in the art.

In general, the formation of the resistance domain 38 on a surface requires a solvent system in order to dissolve the polymer prior to film formation thereon. In one preferred embodiment, acetone and ethanol are used to dissolve cellulose acetate; however one skilled in the art appreciates numerous solvent systems that are available for cellulosic polymers (and other polymers), for example acetone and water can be used. Additionally, one skilled in the art appreciates that the relative amounts of solvent may be dependent upon the cellulosic polymer (or other polymer) used, its molecular weight, its method of deposition, and its desired thickness. However, in preferred embodiments, a percent solute from about 1% to about 10% can be used (to form the resistance domain solution) to produce the desired result, namely, the resistance domain of the preferred embodiments. In fact, it is understood that the cellulosic based polymer (or other polymer) used, its molecular weight, method of deposition, hydroxyl content, acetyl content, and desired thickness are all parameters that may vary depending on one or more other of the parameters, and can be varied accordingly as is appreciated by one skilled in the art.

In preferred embodiments, the resistance domain 38 is deposited onto the enzyme domain 36 to yield a domain thickness of from about 0.05 micron or less to about 20 microns or more, more preferably from about 0.05, 0.1, 0.15, 0.2, 0.25, 0.3, 0.35, 0.4, 0.45, 0.5, 1, 1.5, 2, 2.5, 3, or 3.5 to about 4, 5, 6, 7, 8, 9, 10, 11, 12, 13, 14, 15, 16, 17, 18, 19, or 19.5 microns, and more preferably from about 2, 2.5 or 3 microns to about 3.5, 4, 4.5, or 5 microns. Preferably, the resistance domain is deposited onto the enzyme domain by spray coating or dip coating.

In general, the membrane system of the preferred embodiments can be formed and/or deposited on the exposed electroactive surfaces (e.g., at least the working and reference electrodes) using known thin film techniques (for example, casting, spray coating, drawing down, vapor deposition, electro-depositing, dip coating, and the like). Preferably, the resistance domain 38 is deposited by spray or dip coating. In some embodiments, the resistance domain 38 is formed by dip coating one or more times (namely, forming one or more of the same or different layers) in a coating solution(s). In embodiments wherein dip coating is used to deposit the resistance domain at room temperature, a preferred insertion rate of from about 10 inches per minute to about 100 inches per minute, with a preferred dwell time of from about 0 seconds to about 10 seconds, and a preferred withdrawal rate of from about 10 inch per minute to about 100 inches per minute provide a functional coating. Suitable cure time ranges from about 1 minute to about 30 minutes, and more preferably about 3 minutes, at room temperature (and can be accomplished under vacuum (e.g., 20 to 30 in Hg)). However, values outside of those set forth above can be acceptable or even desirable in certain embodiments, for example, dependent upon viscosity and surface tension as is appreciated by one skilled in the art. In one exemplary embodiment of needle-type (transcutaneous) sensor such as described with reference to FIGS. 1B and 2B, the resistance domain 38 is formed by dip coating the sensor into an resistance domain solution using an insertion rate of about 40 inches/min., a dwell time of less than about 1 second, and a withdrawal rate of about 40 inches/min., and curing the domain for from about 3 minutes at room temperature. While not wishing to be bound by theory, it is believed that a fast dip rate as described above advantageously provides the necessary membrane coating while minimizing exposure of the enzyme domain to the solvent used in the resistance domain solution, which has been known to inactivate the enzyme in some prior art sensors. The dip process may be repeated from about 1 to 10 times or more, preferably about 3 times, to form an appropriate domain thickness (e.g., about 1 to about 5 microns). However, the resistance domain can be formed using any known method as will be appreciated by one skilled in the art.

Additionally, in some embodiments, wherein the sensor is configured for implantation into host tissue, the resistance domain is configured to interface with the host tissue and to provide a bioprotective barrier that protects the sensor from cellular invasion. While not wishing to be bound by theory, it is believed that many cellulosic polymers provide sufficient biodurability to protect the host tissue from the underlying membrane system (and vice versa).

Electrode Domain

In some embodiments, the membrane system comprises an optional electrode domain (not shown). The electrode domain is provided to ensure that an electrochemical reaction occurs between the electroactive surfaces of the working electrode and the reference electrode, and thus the electrode domain is preferably situated adjacent to the electroactive surfaces. Preferably, the electrode domain includes a semipermeable coating that maintains a layer of water at the electrochemically reactive surfaces of the sensor, for example, a humectant in a binder material can be employed as an electrode domain; this allows for the full transport of ions in the aqueous environment. The electrode domain can also assist in stabilizing the operation of the sensor by overcoming electrode start-up and drifting problems caused by inadequate electrolyte. The material that forms the electrode domain can also protect against pH-mediated damage that can result from the formation of a large pH gradient due to the electrochemical activity of the electrodes.

In one embodiment, the electrode domain includes a flexible, water-swellable, hydrogel film having a “dry film” thickness of from about 0.05 micron or less to about 20 microns or more, more preferably from about 0.05, 0.1, 0.15, 0.2, 0.25, 0.3, 0.35, 0.4, 0.45, 0.5, 1, 1.5, 2, 2.5, 3, or 3.5 to about 4, 5, 6, 7, 8, 9, 10, 11, 12, 13, 14, 15, 16, 17, 18, 19, or 19.5 microns, and more preferably from about 2, 2.5 or 3 microns to about 3.5, 4, 4.5, or 5 microns. “Dry film” thickness refers to the thickness of a cured film cast from a coating formulation by standard coating techniques.

In certain embodiments, the electrode domain is formed of a curable mixture of a urethane polymer and a hydrophilic polymer. Particularly preferred coatings are formed of a polyurethane polymer having carboxylate functional groups and non-ionic hydrophilic polyether segments, wherein the polyurethane polymer is crosslinked with a water soluble carbodiimide (e.g., 1-ethyl-3-(3-dimethylaminopropyl)carbodiimide (EDC))) in the presence of polyvinylpyrrolidone and cured at a moderate temperature of about 50° C.

Preferably, the electrode domain is deposited by spray or dip-coating the electroactive surfaces of the sensor. More preferably, the electrode domain is formed by dip-coating the electroactive surfaces in an electrode solution and curing the domain for a time of from about 15 to about 30 minutes at a temperature of from about 40 to about 55° C. (and can be accomplished under vacuum (e.g., 20 to 30 in Hg)). In embodiments wherein dip-coating is used to deposit the electrode domain, a preferred insertion rate of from about 1 to about 3 inches per minute, with a preferred dwell time of from about 0.5 to about 2 minutes, and a preferred withdrawal rate of from about 0.25 to about 2 inches per minute provide a functional coating. However, values outside of those set forth above can be acceptable or even desirable in certain embodiments, for example, dependent upon viscosity and surface tension as is appreciated by one skilled in the art. In one embodiment, the electroactive surfaces of the electrode system are dip-coated one time (one layer) and cured at 50° C. under vacuum for 20 minutes.

Although an independent electrode domain is described herein, in some embodiments, sufficient hydrophilicity can be provided in the interference domain and/or enzyme domain (e.g., the domain adjacent to the electroactive surfaces) so as to provide for the full transport of ions in the aqueous environment (e.g. without a distinct electrode domain).

Oxygen Conduit

As described above, certain sensors depend upon an enzyme within the membrane system through which the host's bodily fluid passes and in which the analyte (for example, glucose) within the bodily fluid reacts in the presence of a co-reactant (for example, oxygen) to generate a product. The product is then measured using electrochemical methods, and thus the output of an electrode system functions as a measure of the analyte. For example, when the sensor is a glucose oxidase based glucose sensor, the species measured at the working electrode is H₂O₂. An enzyme, glucose oxidase, catalyzes the conversion of oxygen and glucose to hydrogen peroxide and gluconate according to the following reaction:
Glucose+O₂→Gluconate+H₂O₂

Because for each glucose molecule reacted there is a proportional change in the product, H₂O₂, one can monitor the change in H₂O₂ to determine glucose concentration. Oxidation of H₂O₂ by the working electrode is balanced by reduction of ambient oxygen, enzyme generated H₂O₂ and other reducible species at a counter electrode, for example. See Fraser, D. M., “An Introduction to In vivo Biosensing: Progress and Problems.” In “Biosensors and the Body,” D. M. Fraser, ed., 1997, pp. 1-56 John Wiley and Sons, New York))

In vivo, glucose concentration is generally about one hundred times or more that of the oxygen concentration. Consequently, oxygen is a limiting reactant in the electrochemical reaction, and when insufficient oxygen is provided to the sensor, the sensor is unable to accurately measure glucose concentration. Thus, depressed sensor function or inaccuracy is believed to be a result of problems in availability of oxygen to the enzyme and/or electroactive surface(s).

Accordingly, in an alternative embodiment, an oxygen conduit (for example, a high oxygen solubility domain formed from silicone or fluorochemicals) is provided that extends from the ex vivo portion of the sensor to the in vivo portion of the sensor to increase oxygen availability to the enzyme. The oxygen conduit can be formed as a part of the coating (insulating) material or can be a separate conduit associated with the assembly of wires that forms the sensor.

Porous Biointerface Materials

In alternative embodiments, the distal portion 42 includes a porous material disposed over some portion thereof, which modifies the host's tissue response to the sensor. In some embodiments, the porous material surrounding the sensor advantageously enhances and extends sensor performance and lifetime in the short term by slowing or reducing cellular migration to the sensor and associated degradation that would otherwise be caused by cellular invasion if the sensor were directly exposed to the in vivo environment. Alternatively, the porous material can provide stabilization of the sensor via tissue ingrowth into the porous material in the long term. Suitable porous materials include silicone, polytetrafluoroethylene, expanded polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polyvinyl alcohol (PVA), polybutylene terephthalate (PBT), polymethylmethacrylate (PMMA), polyether ether ketone (PEEK), polyamides, polyurethanes, cellulosic polymers, polysulfones and block copolymers thereof including, for example, di-block, tri-block, alternating, random and graft copolymers, as well as metals, ceramics, cellulose, hydrogel polymers, poly (2-hydroxyethyl methacrylate, pHEMA), hydroxyethyl methacrylate, (HEMA), polyacrylonitrile-polyvinyl chloride (PAN-PVC), high density polyethylene, acrylic copolymers, nylon, polyvinyl difluoride, polyanhydrides, poly(1-lysine), poly (L-lactic acid), hydroxyethylmethacrylate, hydroxyapeptite, alumina, zirconia, carbon fiber, aluminum, calcium phosphate, titanium, titanium alloy, nintinol, stainless steel, and CoCr alloy, or the like, such as are described in U.S. Publ. No. US-2005-0031689-A1 and U.S. Publ. No. US-2005-0112169-A1.

In some embodiments, the porous material surrounding the sensor provides unique advantages in the short term (e.g., one to 14 days) that can be used to enhance and extend sensor performance and lifetime. However, such materials can also provide advantages in the long term too (e.g., greater than 14 days). Particularly, the in vivo portion of the sensor (the portion of the sensor that is implanted into the host's tissue) is encased (partially or fully) in a porous material. The porous material can be wrapped around the sensor (for example, by wrapping the porous material around the sensor or by inserting the sensor into a section of porous material sized to receive the sensor). Alternately, the porous material can be deposited on the sensor (for example, by electrospinning of a polymer directly thereon). In yet other alternative embodiments, the sensor is inserted into a selected section of porous biomaterial. Other methods for surrounding the in vivo portion of the sensor with a porous material can also be used as is appreciated by one skilled in the art.

The porous material surrounding the sensor advantageously slows or reduces cellular migration to the sensor and associated degradation that would otherwise be caused by cellular invasion if the sensor were directly exposed to the in vivo environment. Namely, the porous material provides a barrier that makes the migration of cells towards the sensor more tortuous and therefore slower (providing short term advantages). It is believed that this reduces or slows the sensitivity loss normally observed in a short-term sensor over time.

In an embodiment wherein the porous material is a high oxygen solubility material, such as porous silicone, the high oxygen solubility porous material surrounds some of or the entire in vivo portion 42 of the sensor. High oxygen solubility materials are materials that dynamically retain a high availability of oxygen that can be used to compensate for the local oxygen deficit during times of transient ischemia (e.g., silicone and fluorocarbons). It is believed that some signal noise normally seen by a conventional sensor can be attributed to an oxygen deficit. In one exemplary embodiment, porous silicone surrounds the sensor and thereby effectively increases the concentration of oxygen local (proximal) to the sensor. Thus, an increase in oxygen availability proximal to the sensor as achieved by this embodiment ensures that an excess of oxygen over glucose is provided to the sensor; thereby reducing the likelihood of oxygen limited reactions therein. Accordingly, by providing a high oxygen solubility material (e.g., porous silicone) surrounding the in vivo portion of the sensor, it is believed that increased oxygen availability, reduced signal noise, longevity, and ultimately enhanced sensor performance can be achieved.

Bioactive Agents

In some alternative embodiments, a bioactive agent is incorporated into the above described porous material and/or membrane system, such as is described in U.S. Publ. No. US-2005-0031689-A1, which diffuses out into the environment adjacent to the sensing region. Additionally or alternately, a bioactive agent can be administered locally at the exit-site or implantation-site. Suitable bioactive agents are those that modify the host's tissue response to the sensor, for example anti-inflammatory agents, anti-infective agents, anesthetics, inflammatory agents, growth factors, immunosuppressive agents, antiplatelet agents, anti-coagulants, anti-proliferates, ACE inhibitors, cytotoxic agents, anti-barrier cell compounds, vascularization-inducing compounds, anti-sense molecules, or mixtures thereof, such as are described in more detail in U.S. Publ. No. US-2005-0031689-A1.

In embodiments wherein the porous material is designed to enhance short-term (e.g., from about 1 to about 14 days) lifetime or performance of the sensor, a suitable bioactive agent can be chosen to ensure that tissue ingrowth does not substantially occur within the pores of the porous material. Namely, by providing a tissue modifying bioactive agent, such as an anti-inflammatory agent (for example, Dexamethasone), substantially tissue ingrowth can be inhibited, at least in the short term, in order to maintain sufficient glucose transport through the pores of the porous material to maintain a stable sensitivity.

In embodiments wherein the porous material is designed to enhance long-term (e.g., from about a day to about a year or more) lifetime or performance of the sensor, a suitable bioactive agent, such as a vascularization-inducing compound or anti-barrier cell compound, can be chosen to encourage tissue ingrowth without barrier cell formation.

Membrane Architectures

Although the illustrated embodiment describes one preferred membrane architecture, namely, an interference domain proximal to the electroactive surfaces, an enzyme domain more distal to the electroactive surfaces than the interference domain, and a resistance domain more distal to the electroactive surfaces than the enzyme domain, a variety of alternative membrane architectures can be utilized. In one alternative embodiment, the enzyme domain is proximal to the electroactive surfaces and the interference domain is more distal to the electroactive surfaces than the enzyme domain. In another alternative embodiment, the enzyme domain is proximal to the electroactive surfaces and the interference domain is more distal to the electroactive surfaces than the enzyme domain as described above, however an electrode domain is located between the enzyme domain and the electroactive surfaces. In another alternative embodiment, an interference domain is more distal to the electroactive surfaces than the resistance domain. In yet another alternative embodiment, an interference domain is more distal to the electrode surface than the enzyme domain. In fact, a variety of alternative membrane configurations are possible within the scope of the preferred embodiments, as is appreciated by one skilled in the art.

In certain embodiments, a variable frequency microwave oven can be used to cure the membrane domains/layers. In general, microwave ovens directly excite the rotational mode of solvents. Consequently, microwave ovens cure coatings from the inside out rather than from the outside in as with conventional convection ovens. This direct rotational mode excitation is responsible for the typically observed “fast” curing within a microwave oven. In contrast to conventional microwave ovens, which rely upon a fixed frequency of emission that can cause arcing of dielectric (metallic) substrates if placed within a conventional microwave oven, Variable Frequency Microwave (VFM) ovens emit thousands of frequencies within 100 milliseconds, which substantially eliminates arcing of dielectric substrates. Consequently, the membrane domains/layers can be cured even after deposition on metallic electrodes as described herein. While not wishing to be bound by theory, it is believe that VFM curing can increase the rate and completeness of solvent evaporation from a liquid membrane solution applied to a sensor, as compared to the rate and completeness of solvent evaporation observed for curing in conventional convection ovens.

In certain embodiments, VFM is can be used together with convection oven curing to further accelerate cure time. In some sensor applications wherein the membrane is cured prior to application on the electrode (see, for example, U.S. Publication No. US-2005-0245799-A1), conventional microwave ovens (e.g., fixed frequency microwave ovens) can be used to cure the membrane layer.

Treatment of Resistance Domain/Membrane System

Although the above-described membrane systems generally include a curing step in formation of the membrane system, including the resistance domain, some alternative embodiments further include an additional treatment step, which may be performed directly after the formation of the resistance domain and/or some time after the formation of the entire membrane system (and anytime in between). In some embodiments, the additional treatment step is performed during (in combination with) sterilization of the sensor.

In some embodiments, the membrane system (or resistance domain) is treated by exposure to ionizing radiation, for example electron beam radiation, UV radiation, X-ray radiation, gamma radiation, and the like. While not wishing to be bound by theory, it is believed that exposing the resistance domain to ionizing radiation substantially cross links the resistance domain and thereby creates a tighter, less permeable network than a resistance domain that has not been exposed to ionizing radiation.

In some embodiments, the membrane system (or resistance domain) is crosslinked by forming free radicals, which may include the use of ionizing radiation, thermal initiators, chemical initiators, photoinitiators (e.g., UV and visible light), and the like. Any suitable initiator or any suitable initiator system can be employed, for example, α-hydroxyketone, α-aminoketone, ammonium persulfate (APS), redox systems such as APS/bisulfite, or potassium permanganate. Suitable thermal initiators include but are not limited to potassium persulfate, ammonium persulfate, sodium persulfate, and mixtures thereof.

While not wishing to be bound by theory, it is believed that cross linking the resistance domain to form free radicals effectively forms covalent bonds that create a tighter, less permeable network than a resistance domain that has not been cross linked.

In embodiments wherein electron beam radiation is used to treat the membrane system (or resistance domain), a preferred exposure time is from about 6 Kilo-Gray (kGy) or about 12 kGy to about 25 kGy or about 50 kGy, and more preferably 25 kGy. However, one skilled in the art can appreciate that choice of molecular weight, composition of cellulosic polymer (or other polymer) and/or thickness of layer can affect the preferred exposure time of membrane to radiation. Preferably, the exposure is sufficient for substantially cross linking the resistance domain to form free radicals, but does not destroy or break down the membrane or does not damage the underlying electroactive surfaces.

In embodiments wherein UV radiation is employed to treat the membrane, UV rays from about 200 nm to about 400 nm are preferred; however values outside of this range are possible dependent upon the cellulosic polymer and/or other polymer used.

In general, sterilization of the transcutaneous sensor can be completed after final assembly, utilizing methods such as electron beam radiation, gamma radiation, glutaraldehyde treatment, or the like. The sensor can be sterilized prior to or after packaging. In an alternative embodiment, one or more sensors can be sterilized using variable frequency microwave chamber(s), which can increase the speed and reduce the cost of the sterilization process. In another alternative embodiment, one or more sensors can be sterilized using ethylene oxide (EtO) gas sterilization, for example, by treating with 100% ethylene oxide, which can be used when the sensor electronics are not detachably connected to the sensor and/or when the sensor electronics must undergo a sterilization process. In one embodiment, one or more packaged sets of transcutaneous sensors (e.g., 1, 2, 3, 4, or 5 sensors or more) are sterilized simultaneously.

Experiments

The following examples serve to illustrate certain preferred embodiments and aspects and are not to be construed as limiting the scope thereof.

### Transcutaneous Glucose Sensor with Cellulose Acetate Resistance Domain

A short term (transcutaneous) sensor was generally built by providing a platinum wire, vapor-depositing the platinum with Parylene to form an insulating coating, helically winding a silver wire around the insulated platinum wire (to form a “twisted pair”), masking sections of electroactive surface of the silver wire, vapor-depositing Parylene on the twisted pair, chloridizing the silver electrode to form silver chloride reference electrode, and removing a radial window on the insulated platinum wire to expose a circumferential electroactive working electrode surface area thereon, this assembly also referred to as a “parylene-coated twisted pair assembly” (which is described in more detail in U.S. Publ. No. US-2006-0020187-A1).

An interference domain was formed on the parylene-coated twisted pair assembly by dip coating in an interference domain solution comprising 7 weight percent, 50,000 molecular weight cellulose acetate (Aldrich 419028 St. Louis, Mo.) in a 2:1 acetone/ethanol solvent solution, followed by drying at room temperature for 3 minutes. This interference domain solution dip coating step was repeated three more times to form an interference domain comprised of four layers of cellulose acetate on the assembly. The dip length (insertion depth) was adjusted to ensure that the cellulose acetate covered from the tip of the working electrode, over the exposed electroactive working electrode window, to cover a distal portion of the exposed electroactive reference electrode.

An enzyme domain was formed over the interference domain by subsequently dip coating the assembly in an enzyme domain solution and drying in a vacuum oven for 20 minutes at 50° C. This dip coating process was repeated once more to form an enzyme domain comprised of two layers.

A resistance domain was formed over the enzyme domain using a resistance domain solution comprising 7 weight percent, 38,000 molecular weight cellulose acetate (Eastman CA-320S, Kingsport, Tenn.) in a 90:10 acetone/water solvent solution, followed by drying at room temperature for 3 minutes. The cellulose acetate used in this test included an acetyl content of about 32.0 wt % and a hydroxyl content of about 8.7 wt %. This resistance domain solution dip coating step was repeated two more times to form a resistance domain comprised of three layers of cellulose acetate on the assembly.

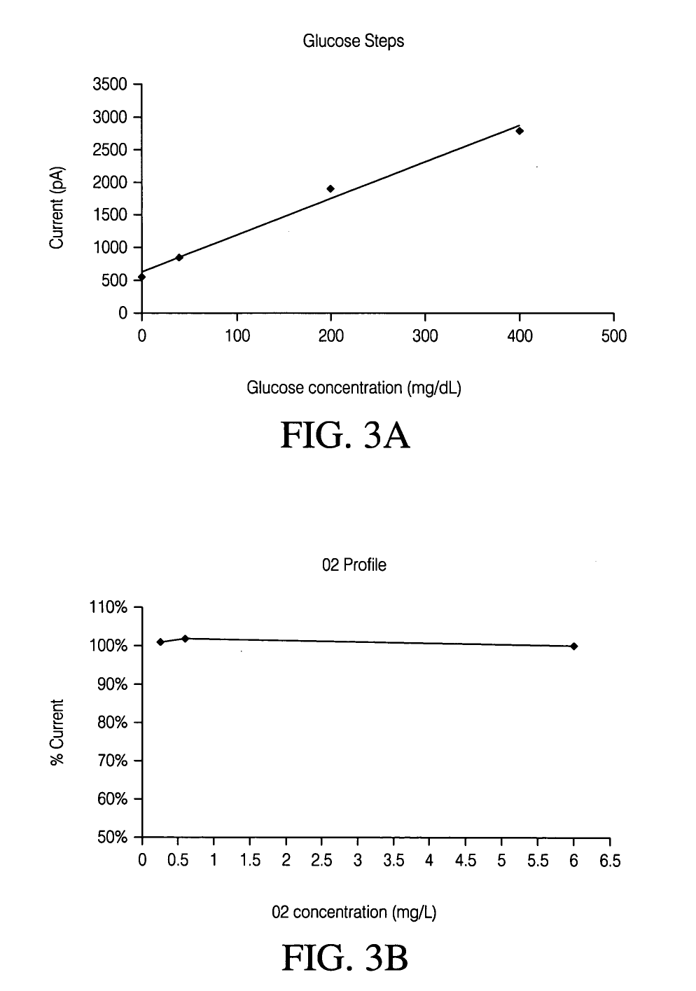

In vitro tests were run to evaluate the ability of the sensor to pass glucose and oxygen. Namely, the sensor was immersed in 40, 200, and 400 mg/dL glucose solutions, and subsequently in 6, 0.6, and 0.25 mg/L oxygen solutions while immersed in 400 mg/dL glucose, while its electrical signal was monitored. The results are shown in FIGS. 3A to 3B.

FIG. 3A is a graph that illustrates sensor's response to each glucose solution (40, 200, and 400 mg/dL glucose steps). The x-axis represents glucose in mg/dL; the y-axis represents current in picoAmps. Namely, the sensor's response is plotted for each glucose step and the data points regressed to determine the slope (and intercept) of the sensor. The resulting slope is about 5.5 with an R² value of about 0.9882 showing that the sensor performs well and has a functional slope and excellent linearity.

FIG. 3B is a graph that illustrates sensor's response to each oxygen solution (6, 0.6, and 0.25 mg/L oxygen steps). The x-axis represents oxygen in mg/L; the y-axis represents current as a percentage of signal at 6 mg/L oxygen when a glucose concentration of 400 mg/dL is present. Namely, the sensor's response is plotted for each oxygen step and shows approximately 100% oxygen performance. Accordingly, it is believed that a glucose sensor built with a cellulose acetate resistance domain as described herein is capable of controlling the flux of glucose and oxygen through the resistance domain such that glucose can be accurately measured within clinically relevant ranges (e.g., about 40 to about 400 mg/dL).

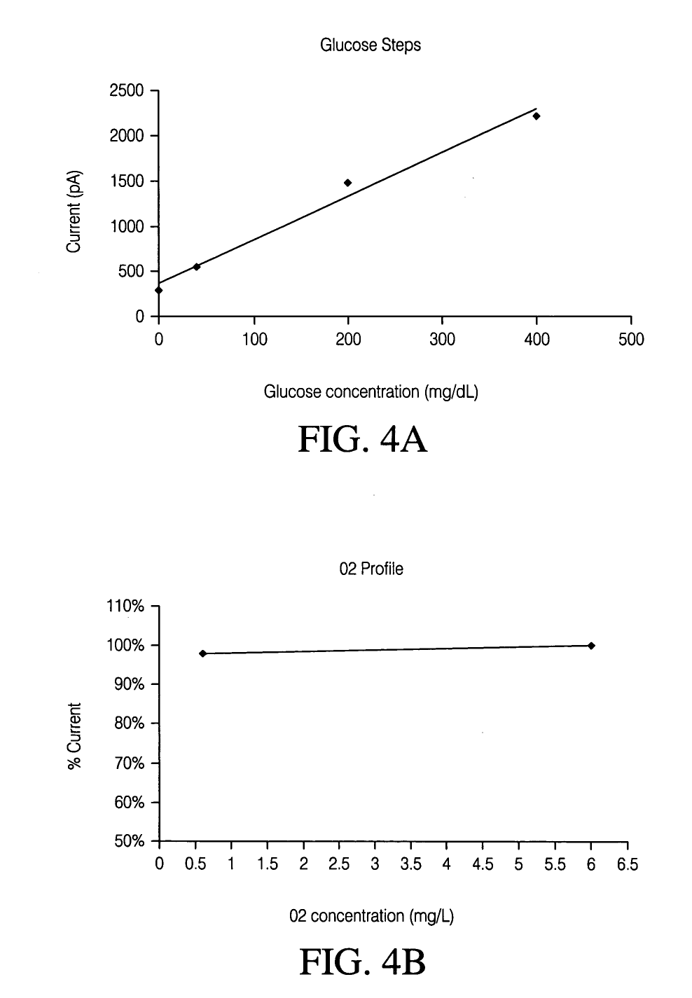

Subsequently, the above-described sensor was exposed to 25 kGy electron beam radiation, and the sensor was again immersed in 40, 200 and 400 mg/dL glucose solutions, and subsequently in 6, 0.6, and 0.25 mg/L oxygen solutions while in 400 mg/dL glucose, while its electrical signal was monitored. The results are shown in FIGS. 4A to 4B.

FIG. 4A is a graph that illustrates sensor's response to each glucose solution (40, 200, and 400 mg/dL glucose steps). The x-axis represents glucose in mg/dL; the y-axis represents current in picoAmps. Namely, the sensor's response is plotted for each glucose step and the data points regressed to determine the slope (and intercept) of the sensor. The resulting slope is about 4.8 with an R² value of about 0.9849 showing that the sensor performance is substantially sustained after electron beam exposure. While not wishing to be bound by theory, it is believed that the slight decrease in slope after electron beam irradiation may be caused by cross linking of the cellulose acetate, which is believed to form a tighter, less permeable network.

FIG. 4B is a graph that illustrates sensor's response to each oxygen solution (6 and 0.6 mg/L oxygen steps). The x-axis represents oxygen in mg/L; the y-axis represents current as a percentage of signal at 6 mg/L oxygen when a glucose concentration of 400 mg/dL is present. Namely, the sensor's response is plotted for each oxygen step and shows approximately 100% oxygen performance down to 0.6 mg/L.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

What is claimed is:

1. An electrochemical analyte sensor for measuring a concentration of an analyte in a host, the sensor comprising an electroactive surface and a membrane system disposed thereon, wherein the membrane system comprises an interference domain configured to resist passage therethrough of at least one interfering species, an enzyme domain configured to catalyze a reaction of the analyte with a co-reactant, and a resistance domain configured to control a flux of the analyte therethrough, wherein the resistance domain comprises a cellulosic polymer.

2. The sensor of claim 1, wherein the interference domain is adjacent to the electroactive surface.

3. The sensor of claim 1, wherein the enzyme domain is more distal to the electroactive surface than the interference domain.

4. The sensor of claim 1, wherein the interference domain is more distal to the electroactive surface than the resistance domain.

5. The sensor of claim 1, wherein the interference domain is more distal to the electroactive surface than the enzyme domain.

6. The sensor of claim 1, wherein the resistance domain is more distal to the electroactive surface than the enzyme domain.

7. The sensor of claim 1, further comprising an electrode domain, wherein the electrode domain is situated between the interference domain and the electroactive surface.

8. The sensor of claim 1, wherein the resistance domain is an ionizing radiation-treated resistance domain, wherein the ionizing radiation is selected from the group consisting of UV, electron beam, gamma, and X-ray radiation.

9. The sensor of claim 1, wherein the cellulosic polymer is selected from the group consisting of cellulose acetate, 2-hydroxyethyl cellulose, cellulose acetate phthalate, cellulose acetate propionate, cellulose acetate butyrate, and cellulose acetate trimellitate.

10. The sensor of claim 1, wherein the cellulosic polymer comprises cellulose acetate.

11. The sensor of claim 10, wherein the cellulose acetate has a molecular weight of less than about 50,000.

12. The sensor of claim 10, wherein the molecular weight of the cellulose acetate is about 38,000.

13. The sensor of claim 10, wherein the cellulose acetate comprises at least about 7 wt. % hydroxyl.

14. The sensor of claim 10, wherein the cellulose acetate comprises about 8.7 wt. % hydroxyl.

15. The sensor of claim 10, wherein the cellulose acetate comprises less than about 35 wt. % acetyl.

16. The sensor of claim 10, wherein the cellulose acetate comprises about 32 wt. % acetyl.

17. The sensor of claim 1, wherein a thickness of the resistance domain is from about 0.05 microns to about 5 microns.

18. The sensor of claim 1, wherein a thickness of the resistance domain is from about 1 to about 3 microns.

19. The sensor of claim 1, wherein the sensor is configured to measure a concentration of glucose.

20. The sensor of claim 1, wherein the sensor is a needle sensor configured for transcutaneous insertion into the host.

21. The sensor of claim 1, wherein the sensor is configured for wholly implanting into the host.

22. The sensor of claim 1, wherein the sensor is configured for implantation into a host tissue, and wherein the resistance domain is configured to interface with the host tissue.

23. The sensor of claim 1, wherein the resistance domain is a bioprotective barrier configured to protect the sensor from cellular invasion.

24. The sensor of claim 1, wherein the membrane system is an ionizing radiation-treated membrane system.

25. The sensor of claim 1, wherein the sensor is an ionizing radiation-treated sensor.

26. An electrochemical analyte sensor for measuring an analyte in a host, the sensor comprising an electroactive surface and a membrane system disposed thereon, wherein the membrane system comprises a resistance domain configured to control a flux of an analyte therethrough, wherein the resistance domain comprises cellulose acetate comprising at least about 7 wt. % hydroxyl.

27. The sensor of claim 26, wherein the cellulose acetate comprises about 8.7 wt. % hydroxyl.

28. The sensor of claim 26, wherein the resistance domain is an ionizing radiation-treated resistance domain, wherein the ionizing radiation is selected from the group consisting of UV, electron beam, gamma, and X-ray radiation.

29. The sensor of claim 26, wherein the cellulose acetate has a molecular weight of less than about 50,000.

30. The sensor of claim 26, wherein a molecular weight of the cellulose acetate is about 38,000.

31. The sensor of claim 26, wherein the cellulose acetate comprises less than about 35 wt. % acetyl.

32. The sensor of claim 26, wherein the cellulose acetate comprises about 32 wt. % acetyl.

33. The sensor of claim 26, wherein a thickness of the resistance domain is from about 0.05 microns to about 5 microns.

34. The sensor of claim 26, wherein a thickness of the resistance domain is from about 1 to about 3 microns.

35. The sensor of claim 26, wherein the membrane system further comprises an enzyme domain configured to catalyze a reaction with the analyte and a co-reactant.

36. The sensor of claim 26, wherein the enzyme domain comprises glucose oxidase.

37. The sensor of claim 26, wherein the membrane system further comprises an interference domain configured to resist passage therethrough of at least one interfering species.

38. The sensor of claim 26, wherein the electroactive surface comprises a working electrode surface and a reference electrode surface, and wherein the interference domain is adjacent to the working reference electrode surface and the reference electrode surface.

39. The sensor of claim 26, wherein the membrane system further comprises an electrode domain.

40. The sensor of claim 26, wherein the sensor is configured to measure a concentration of glucose.

41. The sensor of claim 26, wherein the sensor is a needle sensor configured for transcutaneous insertion into the host.

42. The sensor of claim 26, wherein the sensor is configured for wholly implanting into the host.

43. The sensor of claim 26, wherein the sensor is configured for implantation into a host tissue, and wherein the resistance domain is configured to interface with the host tissue.

44. The sensor of claim 26, wherein the resistance domain is a bioprotective barrier configured to protect the sensor from cellular invasion.

45. The sensor of claim 26, wherein the membrane system is a variable frequency microwave-sterilized membrane system.

46. The sensor of claim 26, wherein the membrane system is an ionizing radiation-treated membrane system.

47. The sensor of claim 26, wherein the sensor is an ionizing radiation-sterilized sensor.

48. An electrochemical analyte sensor for measuring an analyte in a host, the sensor comprising an electroactive surface and a membrane system disposed thereon, wherein the membrane system comprises a resistance domain configured to control a flux of an analyte therethrough, wherein the resistance domain comprises cellulose acetate comprising less than about 35 wt. % acetyl.

49. The sensor of claim 48, wherein the cellulose acetate comprises about 32 wt. % acetyl.

50. The sensor of claim 48, wherein the resistance domain is an ionizing radiation-treated resistance domain, wherein the ionizing radiation is selected from the group consisting of UV, electron beam, gamma, and X-ray radiation.

51. The sensor of claim 48, wherein the cellulose acetate has a molecular weight of less than about 50,000.

52. The sensor of claim 48, wherein a molecular weight of the cellulose acetate is about 38,000.

53. The sensor of claim 48, wherein the cellulose acetate comprises at least about 7 wt. % hydroxyl.

54. The sensor of claim 48, wherein the cellulose acetate comprises about 8.7 wt. % hydroxyl.

55. The sensor of claim 48, wherein a thickness of the resistance domain is from about 0.05 microns to about 5 microns.

56. The sensor of claim 48, wherein a thickness of the resistance domain is from about 1 to about 3 microns.

57. The sensor of claim 48, wherein the membrane system further comprises an enzyme domain configured to catalyze a reaction with the analyte and a co-reactant.

58. The sensor of claim 48, wherein the enzyme domain comprises glucose oxidase.

59. The sensor of claim 48, wherein the membrane system further comprises an interference domain configured to resist passage therethrough of at least one interfering species.

60. The sensor of claim 48, wherein the electroactive surface comprises a working electrode surface and a reference electrode surface, and wherein the interference domain is adjacent to the working reference electrode surface and the reference electrode surface.

61. The sensor of claim 48, wherein the membrane system further comprises an electrode domain.

62. The sensor of claim 48, wherein the sensor is configured to measure a concentration of glucose.

63. The sensor of claim 48, wherein the sensor is a needle sensor configured for transcutaneous insertion into the host.

64. The sensor of claim 48, wherein the sensor is configured for wholly implanting into the host.

65. The sensor of claim 48, wherein the sensor is configured for implantation into a host tissue, and wherein the resistance domain is configured to interface with the host tissue.

66. The sensor of claim 48, wherein the resistance domain is a bioprotective barrier configured to protect the sensor from cellular invasion.

67. The sensor of claim 48, wherein the membrane system is an ionizing radiation-treated membrane system.

68. The sensor of claim 48, wherein the sensor is an ionizing radiation-sterilized sensor.
